(19) United States
(12) Patent Application Publication
(10) Pub. No.: US 2017/0079835 A1
(43) Pub. Date: Mar. 23, 2017

(54) Chemical Thermal Pack and Method

(71) Applicant: Michael Jones, Rathdrum, ID (US)

(72) Inventor: Michael Jones, Rathdrum, ID (US)

(21) Appl. No.: 15/272,236

(22) Filed: Sep. 21, 2016

Related U.S. Application Data
(60) Provisional application No. 62/284,104, filed on Sep. 21, 2015.

(51) Int. Cl. A61F 7/02; A61F 7/08; A61F 7/03

(52) U.S. Cl. CPC A61F 7/0241; A61F 7/03; A61F 7/086; A61F 2007/022

(57) ABSTRACT

A chemical thermal pack is provided having a flexible liquid impermeable pouch, an endothermic/exothermic producing chemical, and a liquid permeable pouch. The flexible liquid impermeable pouch has a resealable closure edge and a scored tamper seal. There is one of an endothermic and an exothermic producing chemical. The liquid permeable pouch is contained within the liquid impermeable pouch and provides a cavity for containing the endothermic/exothermic producing chemical. A user breaches the scored tamper seal and inserts a fluid solvent within the liquid impermeable pouch and reseals the closure edge. The solvent is reactive with the chemical to impart one of an exothermic reaction and an endothermic reaction. A method is also provided.

### RELATED PATENT DATA

This patent resulted U.S. Provisional Patent Application Ser. No. 62/284,104, which was filed Sep. 21, 2015, and which is hereby incorporated herein by reference.

### TECHNICAL FIELD

This disclosure pertains to instant hot/cold packs. More particularly, this disclosure relates to chemical thermal packs with water as a primary reactant and capable of cooling to approximately 30 degrees Fahrenheit or warming to approximately 110 degrees Fahrenheit in a rapid or nearly instantaneous manner.

### BACKGROUND

Conventional instant cold/hot packs have been implemented as a simple plastic bag filled with dry chemical and a breakable bag consisting of water. Such previously known hot/cold packs suffer from several disadvantages. For example, the weight of the packs is significantly greater than what it could be, considering half of the item is enclosed water and water is often otherwise readily available at the point of injury care. This renders the packs unnecessarily cumbersome in both size and shape as each pack is comprised mostly of a soft plastic bag with loose dry chemical and a liquid pouch. Furthermore, the extra weight and bulk (shape) can negatively affect the durability of an item that has been designed to be broken in order to be activated. As such, these devices are not suitable for use in a great deal of military, outdoor, or other sporting events in which emergency first aid use of hot/cold therapy is most often warranted, but materials are limited. Further, the water creates extreme weather complications with respect to cold weather exposure when storing, shipping, and using expanding fluids under low pressure.

### SUMMARY

A chemical thermal pack is provided having a vacuum sealed plastic instant hot/cold pack comprising a small enclosed mesh/paper pouch consisting of dry chemical for hot/cold therapy. In use, a user can tear open the scored tamper seal, fill the pack with clean water to the fill line indicated on the package instructions, reseal the pack, and shake the pack vigorously until thoroughly mixed. A chemical reaction occurs as the chemical mixes with the added water. Such chemical reaction is capable of rendering chemical reaction cooling to approximately 30 degrees Fahrenheit or rendering chemical reaction warming to approximately 110 degrees Fahrenheit to provide hot or cold therapy for first aid use.

According to one aspect, a chemical thermal pack is provided having a flexible liquid impermeable pouch, an endothermic/exothermic producing chemical, and a liquid permeable pouch. The flexible liquid impermeable pouch has a resealable closure edge and a scored tamper seal. There is an endothermic/exothermic producing chemical. The liquid permeable pouch is contained within the liquid impermeable pouch and provides a cavity for containing the endothermic/exothermic producing chemical. A user breaches the scored tamper seal and inserts a fluid solvent within the liquid impermeable pouch and reseals the closure edge. The solvent is reactive with the chemical to impart one of an exothermic reaction and an endothermic reaction.

According to another aspect, a chemical thermal pack is provided having a pouch body of water-impermeable film, a liquid permeable pouch, and an endothermic/exothermic producing chemical. The pouch body of water-impermeable film has a front wall and a rear wall, at least one border seal configured to extend along at least a portion of a circumferential pouch border, a resealable parting seal configured to connect the front wall and the rear wall to render the pouch selectively sealable, and a scored tamper seal provided on the pouch border outwardly of the parting seal. The liquid [solvent] permeable pouch is contained within the liquid impermeable pouch providing a cavity. The endothermic/exothermic producing chemical is contained within the cavity of the liquid permeable pouch. The tamper seal is configured to be breached by a user, water is introduced through the unsealed parting seal, the parting seal is sealed, and a chemical reaction occurs in the sealed pouch as water passes into the liquid permeable pouch to react with the endothermic/exothermic producing chemical.

According to yet another aspect, a method is provided to rendering a chemical thermal pack. The method includes: providing a liquid impermeable pouch having a resealable parting seal, a scored tamper seal outboard of the parting seal, a liquid permeable pouch contained within the liquid impermeable pouch, and an endothermic/exothermic producing chemical contained within the liquid permeable pouch; separating the scored tamper seal; inserting solvent through the parting seal; sealing the parting seal; and agitating the sealed pack to mix the solvent and the chemical to generate one of an endothermic reaction and an exothermic reaction.

### BRIEF DESCRIPTION OF THE DRAWINGS

FIG. 1 is a perspective view of one version of a chemical thermal pack according to one embodiment illustrating manual removal of the tear away seal.

FIG. 2 is a perspective view of the chemical thermal pack illustrating a user introducing clean water into the opened pack.

FIG. 3 is a front elevational view of the chemical thermal pack of FIGS. 1-2.

### DETAILED DESCRIPTION OF THE ILLUSTRATED EMBODIMENTS

This disclosure of the invention is submitted in furtherance of the constitutional purposes of the U.S. Patent Laws “to promote the progress of science and useful arts” (Article 1, Section 8).

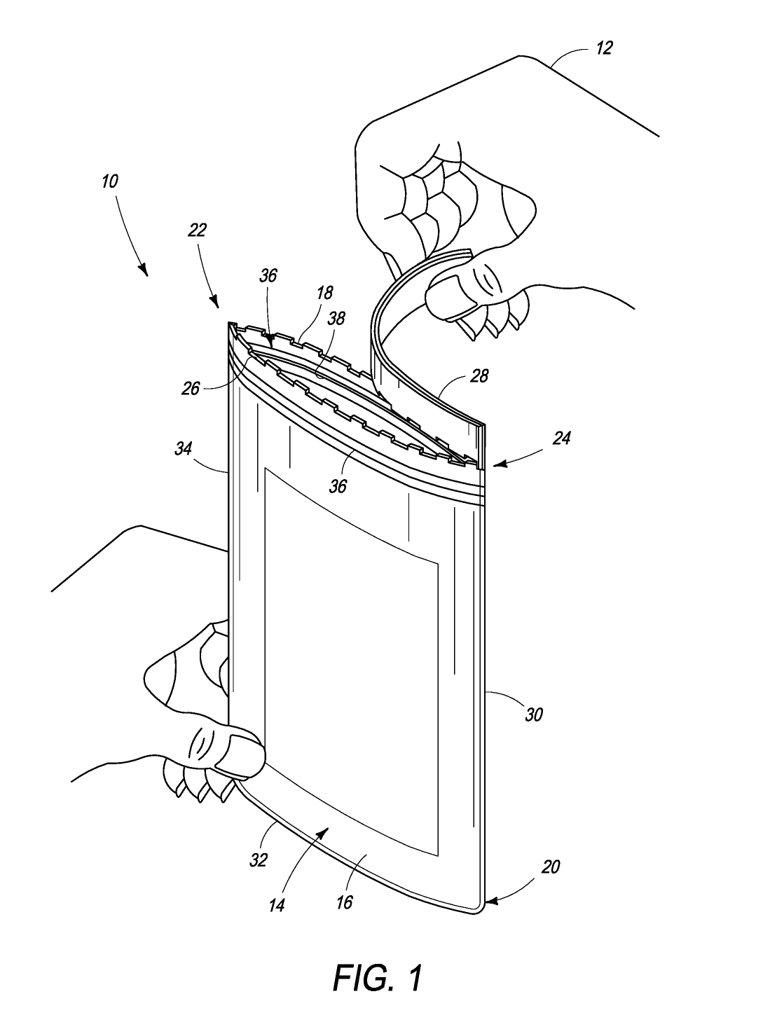

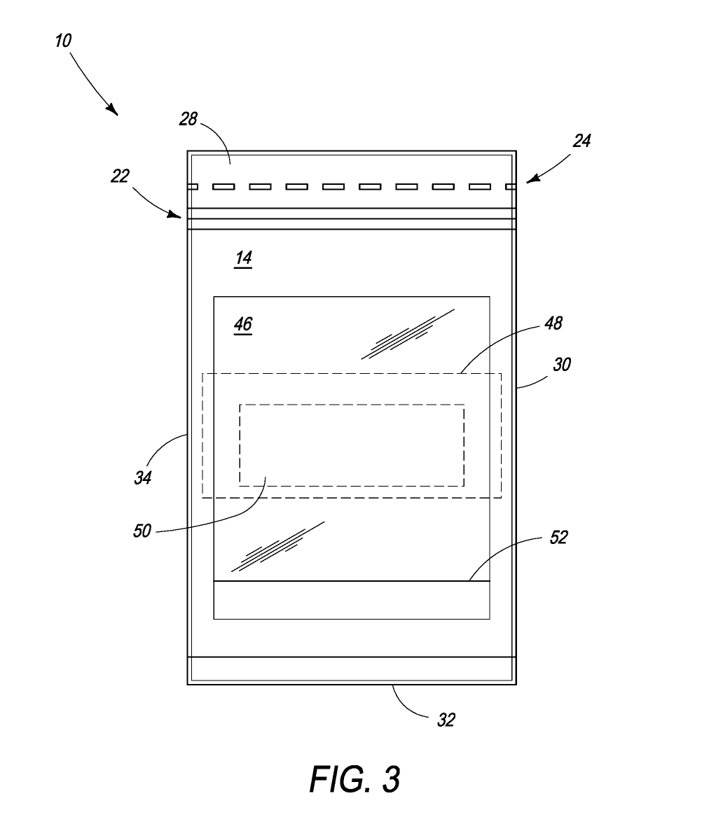

FIG. 1 illustrates a chemical thermal pack, or hot/cold pack 10, according to one implementation. More particularly, pack 10 is illustrated having a flexible liquid impermeable pouch 14, an endothermic/exothermic producing chemical 50 (see FIG. 3), and a liquid permeable pouch 48 (see FIG. 3). The flexible liquid impermeable pouch 14 has a resealable closure edge 22 and a scored tamper seal 24. There is an endothermic/exothermic producing chemical 50. The liquid permeable pouch 48 is contained within the liquid impermeable pouch 14 and provides a cavity for containing the endothermic/exothermic producing chemical 50. A user 12 breaches the scored tamper seal 24 and inserts a fluid solvent 44, such as water, within the liquid impermeable pouch 14 and reseals the closure edge 22. The solvent 44 is reactive with the chemical 50 to impart one of an exothermic reaction and an endothermic reaction.

As shown in FIG. 1, pouch 14 (see FIG. 3) comprises a pouch body of water-impermeable film having a front wall 16 and a rear wall 18, at least one border seal 30, 32, and 34 configured to extend along at least a portion of a circumferential pouch border 20, a resealable parting seal 22 configured to connect the front wall 16 and the rear wall 18 to render the pouch 14 selectively sealable, and a scored tamper seal 24 provided on the pouch border 20 outwardly of the parting seal 24.

As shown in FIGS. 1-3, closure edge 22 according to one implementation, comprises a zip-top bag with a plastic zipper slide seal. Such closure edge is also known as a slider seal, a closure, and a channel and double zipper seal, whereas pouch 14 is also known as a press seal bag, a grip seal bag, and a resealable bag. Optionally, closure edge 22 can be any form of closure including any form of zipper seal, adhesive seal, tape seal, hook and pile (loop) seal, roll seal, banded (metal, plastic, or rubber clip) seal, or a folding seal, or any other means of forming a seal along a bag or container. Similarly, pouch 48 can be closed or formed using such optional closure edge(s).

As used herein, the term “instant” is understood to mean a time span of no more than 15 seconds, or nearly an instant.

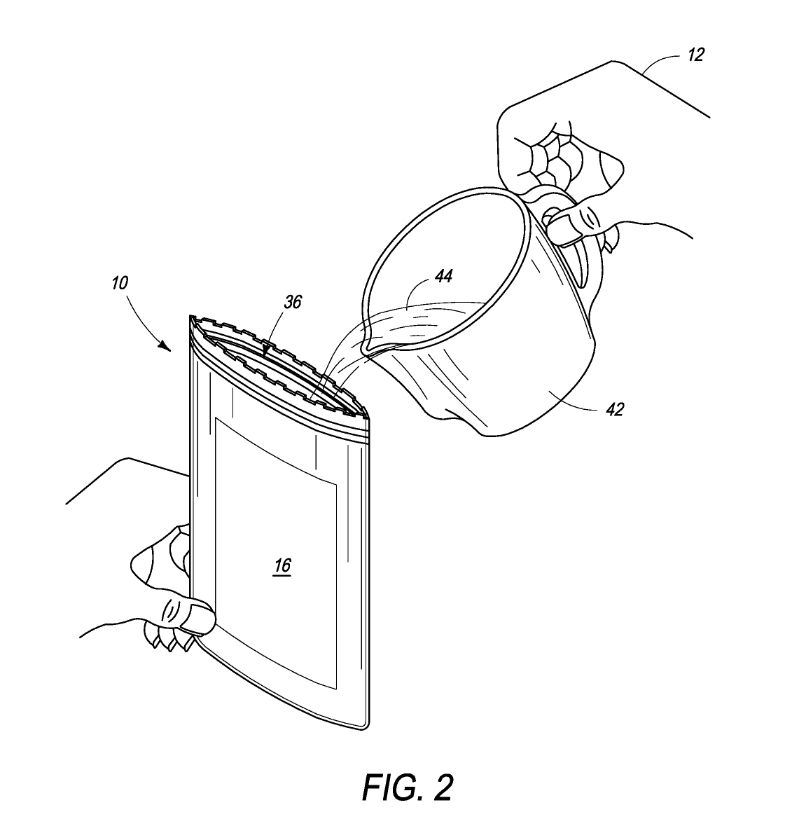

A vacuum sealed plastic instant hot/cold pack 10 comprises a small enclosed mesh/paper pouch 48 (see FIG. 3) housing the chemical reactant 50 for use in either a hot or a cold therapy, depending on the chemical reactant being used. In use, a user 12 tears open the scored tamper seal 24 (see FIG. 1), fills pouch 14 with clean water 44 (see FIG. 2) to a fill line 52 (see FIG. 3) indicated on the package instructions of an exterior label 46 (see FIG. 3). The user then shakes pack 10 vigorously (not shown) until thoroughly mixed. A chemical reaction occurs as the chemical mixes with the added water, or solvent. Such chemical reaction is capable of cooling to approximately 30 degrees Fahrenheit or warming to approximately 110 degrees Fahrenheit to provide hot or cold therapy for first aid use in heat injury, fever, inflammation, soft tissue injury as well as cold injury and shock.

As shown in FIG. 1, pouch 14 (see FIG. 3) of pack 10 comprises a vacuum sealed plastic bag with a scored tamper evident tear away seal 24 located along a top edge that is manually removed by a user 12 during use. Tear away seal 24 comprises a series of equally spaced-apart elongate perforations that form a tear line for separating a top edge strip from the pack. Tear away seal 24 is formed above a zip-top style re-sealable enclosure, or closure edge 22.

According to one implementation, chemical reactant 50 (of FIG. 3) is a dry chemical particle cooling agent consisting of ammonium nitrate, urea or any urea combinations. For the case where it is a dry chemical warming agent, chemical reactant 50 is any combination of calcium chloride. These chemicals, or chemical reactant will be sealed in a plastic mesh and/or a paper envelope having perforations or openings no greater than 800 microns or 0.031 inches, forming liquid permeable pouch 48. Optionally, the chemical reactant 50 can be provided directly within pouch 14 without use of any liquid permeable pouch 48.

In operation the bag is activated for either a cooling or heating reaction which instantly occurs as the chemical mixes with water added to the bag in an amount indicated by a fill line on the package instructions sufficient enough to cause the prescribed chemical reaction. This occurs whether permeable pouch 48 is used or not.

The present embodiment provides a vacuum sealed plastic instant hot/cold pack 10 comprising a small enclosed mesh/paper pouch 48 consisting of dry chemical 50 for hot/cold therapy. In use, a user 12 will tear open the scored tamper seal 24, fill the pouch 14 with clean water 44 from a container (or source) 42 to a fill line 52 indicated on package instructions on an exterior label 46, the user will seal shut the closure edge 22, and the user will shake pack 10 vigorously until thoroughly mixed. A chemical reaction occurs as the chemical mixes with added water. Such chemical reaction is capable of cooling to approximately 30 degrees Fahrenheit or warming to approximately 110 degrees Fahrenheit to provide hot or cold therapy for first aid use in heat injury, fever, inflammation, soft tissue injury as well as cold injury and shock.

As shown in FIG. 3, a front view of the vacuum sealed hot/cold pack 10, according to one construction, comprises a plastic bag of no less than 2 mm thickness and no greater than 4 mm thickness that is constructed and arranged for vacuum seal forming and closure with any commercial use vacuum sealing device with all components being inserted, enclosed and then vacuum sealed within the bag. The bag can be either a top loaded or a bottom loaded bag, and can be of varying shapes and sizes to accommodate different/specified uses. As shown in FIGS. 1-3, the bag (or pouch) 14 is a bottom loaded bag for ease of heat seal after vacuuming, but it could be also be a top loaded bag, wherein the heat seal is placed above, or before, the tear away portion along the top edge.

A shown in FIG. 3, label 46 is a paper and/or plastic adhesive-backed label that is affixed to an exterior of pouch 14 (see FIG. 1). Instructional and safety information, or indicia can be printed on the label and affixed to the pouch, including liquid fill line 52. Optionally, such information can be applied to the pouch by directly printing such information on an exterior surface of the pouch. In such case, fill line 52 is printed directly onto pouch 14. Location of fill line 52 indicates the quantity of solvent, or water needed to be introduced into the pouch for the given solute, or chemical reactant contained in pouch 48. Elevational positioning of fill 52 will depend on the dimensions of pouch 14 and the quantity of solute, or chemical reactant such that a suitable chemical reaction is generated during mixing (or shaking).

As shown in FIG. 3, enclosed chemical pack 48 is made of a heat sealable water permeable plastic, a paper having an integral mesh and/or a bonded insert mesh, or any combination thereof, with holes no greater than 800 microns or 0.031 inches in size for housing of dry chemical particles. Such pack 48 is sized to fit various weights of chemical reactant, either in loose form, or in a cake form.

In one implementation, it is understood that dry particle chemical is used as a reactant to generate an endothermic reaction when mixed with a suitable solvent, as in the case with ammonium nitrate, calcium ammonium nitrate, salts of ammonium nitrate, urea or any urea combinations thereof, including salts of urea. In the case of an exothermic reaction, or warming, the dry particle chemical can be any combination of calcium chloride or salts of calcium chloride. Acid and sugar combinations can also be used. The liquid and the chemical are both reactants in such cases. Weight, or mass of the chemical reactant is determined by size and shape of the plastic bag, and its intended use (and desired heat or cold generation needed for a specific application).

The instructional “Fill Line” the line 52 identified in FIG. 3 on the instructions of label 46 provides a fluid level line elevationally on the packet indicating a measured level for a user to fill the bag with clean water in a 1:1 weight ratio with any of the above named dry chemical particles. Fluid volume, or weight and reactant quantity is determined by the size and shape of the plastic bag and its intended use.

In use, a user 12 of chemical thermal pack 10 will first tear open the scored tamper seal 24 and opens (if not already open) reusable reclose edge 22, as shown in FIG. 1. User 12 will then fill pouch, or pouch body 14 (see FIG. 3) with clean water to the fill line 52 indicated on the package instructions of label 46, as shown in FIG. 2. Next, user 12 will shake pack 10 vigorously until thoroughly mixed (not shown). A resultant chemical reaction occurs as the chemical reactant mixes with the added water, or solvent. Finally, user 12 then applies the chemical thermal pack 10 to an affected area on a patient, or user as desired for therapy in first aid use of heat injury, fever, inflammation, soft tissue injury, as well as cold injury and shock (depending on whether the reactant and solvent used generate an exothermic reaction or an endothermic reaction).

It is important to note that, certain instances, chemical thermal pack 10 is preferably used with an appropriate barrier inserted between chemically activated pack 10 and the skin of a patient or user so as to not create tissue damage from direct thermal to activated pack 10. In use, the pack should be removed if a user/patient feels discomfort and/or pain. The therapy should be alternated in 15-20 minute increments by applying the thermal pack and removing the thermal pack from the patient application site, on and off. It is also important that individuals do not eat or drink contents of the pack. If this occurs, it is important for a user to call a poison control center. Accordingly, a user should keep pack 10 out of reach of children and/or not leave pack 10 unattended, or enable children to operate pack 10 without adult supervision.

Several specific exemplary implementations and uses of the instant hot/cold pack design detailed herein are provided below.

For example, a cold pack implementation of the chemical thermal pack may be used in such first aid and medical emergencies as in the treatment of people and animals afflicted with inflammation, swelling, pain, and fever. Such pack can also be used where other forms of cooling are not practical, such as to provide for a refreshing cool compress for athletes and soldiers. Such pack is also environmentally safe. A heat pack implementation of the chemical thermal pack may be used in such first aid and medical emergencies as in the treatment of people and animals afflicted with cold injuries like chill banes, frost bite, hypothermia, and in the treatment of shock. In both implementations of the chemical thermal pack, high grade of plastic can be used in construction of such packs that is food safe for keeping food/beverages warm/cold as desired for a short period of time (as long as the chemicals do not come into direct contact with consumable items) wherever other forms of refrigeration or warming are not practical and/or warranted. As the chemicals are encased and vacuum sealed, they travel well as a virtually indestructible self contained piece of equipment. They have an almost indefinite shelf life. They are protected from all destructive elements better than with standard instant hot/cold packs. Furthermore, such packs are not subject to solidification due to moisture, heat, or pressure.

In compliance with the statute, the subject matter disclosed herein has been described in language more or less specific as to structural and methodical features. It is to be understood, however, that the claims are not limited to the specific features shown and described, since the means herein disclosed comprise example embodiments. The claims are thus to be afforded full scope as literally worded, and to be appropriately interpreted in accordance with the doctrine of equivalents.

I/we claim,:

1. A chemical thermal pack, comprising:
a flexible liquid impermeable pouch having a resealable closure edge and a scored tamper seal;
an endothermic/exothermic producing chemical; and
a liquid permeable pouch contained within the liquid impermeable pouch providing a cavity for containing the endothermic/exothermic producing chemical;
wherein a user breaches the scored tamper seal and inserts a fluid solvent within the liquid impermeable pouch and reseals the closure edge, the solvent reactive with the chemical to impart one of an exothermic reaction and an endothermic reaction.

2. The chemical thermal pack of claim 1, wherein the pouch comprises a front wall and a rear wall.

3. The chemical thermal pack of claim 2, wherein the pouch further comprises an edge seal provided between the front wall and the rear wall configured to extend along at least a portion of a circumferential pouch border.

4. The chemical thermal pack of claim 3, wherein the resealable closure edge cooperates with the edge seal to completely encompass an entire outer periphery of the pouch and render the pouch with a resealable liquid cavity.

5. The chemical thermal pack of claim 1, wherein the scored tamper seal is provided on the pouch border outwardly of the parting seal.

6. The chemical thermal pack of claim 1, wherein the liquid permeable pouch comprises a heat-sealable water permeable pouch.

7. The chemical thermal pack of claim 6, wherein the pouch has perforations no greater than 800 microns.

8. The chemical thermal pack of claim 1, wherein the endothermic/exothermic chemical comprises one of: A) ammonium nitrate; B) urea; C) a urea combination; and D) a calcium chloride combination.

9. A chemical thermal pack, comprising:
a pouch body of water-impermeable film having a front wall and a rear wall, at least one border seal configured to extend along at least a portion of a circumferential pouch border, a resealable parting seal configured to connect the front wall and the rear wall to render the pouch selectively sealable, and a scored tamper seal provided on the pouch border outwardly of the parting seal;
a liquid permeable pouch contained within the liquid impermeable pouch providing a cavity; and
an endothermic/exothermic producing chemical contained within the cavity of the liquid permeable pouch;
wherein the tamper seal is configured to be breached by a user, water is introduced through the unsealed parting seal, the parting seal is sealed, and a chemical reaction occurs in the sealed pouch as water passes into the liquid permeable pouch to react with the endothermic/exothermic producing chemical.

10. The chemical thermal pack of claim 9, wherein the resealable parting seal cooperates with the border seal to completely encompass an entire outer periphery of the pouch body and render the pouch body with a resealable liquid cavity.

11. The chemical thermal pack of claim 9, wherein the liquid permeable pouch comprises a heat-sealable water permeable pouch.

12. The chemical thermal pack of claim 11, wherein the pouch has perforations no greater than 800 microns.

13. The chemical thermal pack of claim 9, wherein the endothermic/exothermic chemical comprises one of: A) ammonium nitrate; B) urea; C) a urea combination; and D) a calcium chloride combination.

14. The chemical thermal pack of claim 9, wherein the chemical comprises an endothermic chemical comprising one of: A) ammonium nitrate; B) urea; and C) a urea combination.

15. A method of rendering a chemical thermal pack, comprising:
providing a liquid impermeable pouch having a resealable parting seal, a scored tamper seal outboard of the parting seal, a liquid permeable pouch contained within the liquid impermeable pouch, and an endothermic/exothermic producing chemical contained within the liquid permeable pouch;
separating the scored tamper seal;
inserting solvent through the parting seal;
sealing the parting seal; and
agitating the sealed pack to mix the solvent and the chemical to generate one of an endothermic reaction and an exothermic reaction.

16. The method of claim 15, wherein the solvent is water.

17. The method of claim 16, wherein the chemical is a dry chemical particle cooling agent comprising one of: ammonium nitrate, urea or any urea combinations.

18. The method of claim 16, wherein the chemical is a dry chemical particle heating agent comprising one of: a calcium chloride and a combination of calcium chloride.

19. The method of claim 15, wherein the liquid permeable pouch comprises perforations no greater than 800 microns in size.

20. The method of claim 19, wherein the liquid permeable pouch comprises a heat sealable water permeable plastic.
